(19) United States
(12) Patent Application Publication
(10) Pub. No.: US 2006/0165657 A1
(43) Pub. Date: Jul. 27, 2006

(54) Method for delivery of cosmetic by topical application

(76) Inventors: Paul Bernasconi, Chapel Hill, NC (US); William Perry Janzen, Chapel Hill, NC (US); Jennings Franklin Worley III, Carlsbad, CA (US)

Correspondence Address: MYERS BIGEL SIBLEY & SAJOVEC, PO BOX 37428, RALEIGH, NC, 27627, US

(21) Appl. No.: 10/544,790

(22) PCT Filed: Feb. 4, 2004
(86) PCT No.: PCT/US04/03073

§ 371 Date: Feb. 22, 2006

Related U.S. Application Data
(60) Provisional application No. 60/445,209, filed on Feb. 5, 2003.

(51) Int. Cl. A61K 48/00; C12N 15/86

(52) U.S. Cl. 424/93.2; 435/456; 424/239.1

(57) ABSTRACT

The present invention relates to compositions comprising a baculovirus vector and a pharmaceutically acceptable carrier to deliver the baculovirus vector onto or into the skin of a mammal to achieve a cosmetic effect. The present invention also provides methods of producing various cosmetic effects by administering the compositions of this invention onto or into the skin of a mammal.

### RELATED APPLICATIONS

The present application claims the benefit, under 35 U.S.C. §119(e), of U.S. provisional application Ser. No. 60/445,209, filed Feb. 5, 2004, the entire contents of which are incorporated herein by reference.

### FIELD OF INVENTION

The present invention relates to compositions comprising a baculovirus vector and a pharmaceutically acceptable carrier for delivering a heterologous nucleic acid to skin cells to impart a cosmetic effect. Additionally, the present invention relates to methods of delivering nucleic acids to skin cells for cosmetic purposes.

### BACKGROUND OF INVENTION

Women and men generally to wish to appear as young possible and often look to soften the signs of aging of the skin, which are reflected in particular by wrinkles and fine lines. In this respect, advertising and fashion present products intended to retain a radiant and wrinkle-free skin, these being the signs of young skin, for as long as possible, all the more so since physical appearance has an effect on mental attitude and/or on morale. Many equate looks with feeling physically and spiritually young.

Moreover, it has been shown that botulinum toxin, originally used for treating spasms, can affect muscle spasticity conditions (see Blitzer et al., Arch. Otolaiyngol. Head Neck Surg. 119:1018-1022 (1993)) and wrinkles of the glabella, which are intersuperciliary wrinkles (see Carruters et al., J. Derinatol. Sura. Oncol. 18:17-21 (1992)). It is consequently possible, by pharmacological action, to have an effect on the nerve component of wrinkles. Botulinum toxin acts directly at the level of the neuro-muscular junction by blocking the action of acetylcholine on muscular tenseness. Presently, practitioners inject botulinum toxin into the muscle of a subject. This is sometimes a painful procedure. Therefore, it may be advantageous to have a method of administering botulinum toxin to a subject for its cosmetic effects without injections.

The present invention provides methods of expressing a nucleic acid in skin cells via a baculovirus vector for cosmetic purposes. Viruses of the family Baculoviridae (commonly referred to as baculoviruses) have been used to express exogenous nucleic acids in insect cells. One of the most studied baculoviruses is the Autographa californica multiple nuclear polyhedrosis virus (AcMNPV). Although some species of baculoviruses that infect crustacea have been described (Blissard et al., 1990, Ann. Rev. Entomology 35:127), the normal host range of the baculovirus AcMNPV is limited to the order lepidoptera. In the mid-1990s, two groups reported that recombinant baculoviruses containing mammalian cell-active promoters could be used to transduce mammalian cells. Viruses containing either a cytomegalovirus promoter/luciferase gene cassette (Hoffman et al., Proc. Nat. Acad. Sci. U.S.A., 92:10099-10103 (1995)) or a RSV LTR promoter/β-galactosidase cassette (Boyce et al., Proc. Nat. Acad. Sci. U.S.A. 93:2348-2352 (1996)) were tested in primary hepatocytes and a variety of non-hepatic cell lines and it was demonstrated that baculoviruses could transduce mammalian cells.

Current methods of expressing heterologous nucleic acids in a mammalian cell include the use of viral vectors, such as those which are derived from retroviruses, adenoviruses, herpes viruses, vaccinia viruses, polio viruses, alpha viruses, or adeno-associated viruses. Other methods of expressing a heterologous nucleic acid a mammalian cell include direct injection of DNA, the use of ligand-DNA conjugates, the use of adenovirus-ligand-DNA conjugates, calcium phosphate precipitation, electroporation, and methods which utilize a liposome- or polycation-DNA complex. However, the use of a baculovirus vector to express a heterologous nucleic acid in skin cells for cosmetic purposes has not been disclosed or taught by the present art.

### SUMMARY OF THE INVENTION

The present invention provides both compositions and methods comprising the use of a baculovirus vector and a pharmaceutically acceptable carrier for delivery of a baculovirus vector onto, into and/or through the skin of a mammal, wherein the baculovirus vector comprises a promoter operable in a mammalian cell and a heterologous nucleic acid which is expressed to achieve a cosmetic effect.

The heterologous nucleic acid may be delivered to a mammalian target skin cell of a mammal, comprising contacting the epidermis, dermis or subcutaneous tissue of the mammal with the composition comprising the baculovirus vector, under conditions whereby the baculovirus vector is transported onto, into and/or through the epidermis, dermis or subcutaneous tissue and introduced into the target skin cell, where it is expressed.

The present invention also discloses numerous methods of expressing a heterologous nucleic acid in a mammalian skin cell via a baculovirus vector to impart a cosmetic effect, comprising introducing into the mammalian skin cell a baculovirus vector, wherein said baculovirus vector comprises a promoter and a heterologous nucleic acid encoding a gene product that imparts a cosmetic effect in the skin or other cells of the mammal. The baculovirus may be administered topically or subcutaneously.

The methods disclosed by the present invention include a method of reducing skin wrinkles in a mammal, coloring or tanning the skin of a mammal, treating acne in the skin of a mammal, diminishing the appearance of a scar in the skin of a mammal, reducing a fat deposit in the skin of a mammal, reducing perspiration in the skin of a mammal, reducing hair growth in the skin of a mammal, delivering vitamin E to a cell in the skin of a mammal, reducing signs of aging in a mammal, and/or inducing apoptosis in a cell in the skin of a mammal, comprising contacting a composition of the present invention with the skin of a mammal under conditions whereby the baculovirus vector is delivered to a target skin cell, the heterologous nucleic acid is expressed to produce the gene product of the heterologous nucleic acid and the gene product is delivered to an effector cell that produces or imparts or otherwise results in the desired cosmetic effect.

### DETAILED DESCRIPTION OF THE INVENTION

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

The present invention provides a composition comprising a baculovirus vector and a pharmaceutically acceptable carrier used to deliver the baculovirus vector onto, into and/or through the skin of a mammal to deliver a nucleic acid encoding a substance that can impart a cosmetic effect to the mammal. The baculovirus vector preferably comprises a promoter operable in a mammalian cell and a heterologous nucleic acid.

The methods of the present invention utilize recombinant baculoviruses containing mammalian expression cassettes that can be used to transduce mammalian cells. (Kost et al., Curr Opin Biotechnol. 10(5):428-33 (1999); Pfohl et al., Receptors and Channels 8(2):99-111 (2002)). In general, the baculovirus genome is engineered to contain a mammalian cell active promoter, such as the cytomegalovirus promoter, to control gene expression. The transduction of mammalian cells occurs in the absence of viral replication. Specifically, the baculovirus of the present invention is unable to replicate or undergo recombination events with the nucleic acid of the mammalian cell transduced with the baculovirus. The virus is effectively confined to the cell it has entered and dies when this cell dies. This delivery method has been used for the delivery of many genes to a variety of mammalian cells for research purposes, for example, in the form of the BacMam vector (Condreay et al. “Transient and stable gene expression in mammalian cells transduced with a recombinant baculovirus vector” Proc. Natl. Acad. Sci. USA 96:127-132 (1999); Kost and Condreay. “Recombinant baculoviruses as expression vectors for insect and mammalian cells” Current Opinion in Biotechnology 10:428-433 (1999); Kost and Condreay. “Recombinant baculoviruses as mammalian cell gene-delivery vectors” Trends in Biotechnology 20:173-180 (2002); Pfohl et al. “Titration of K_{ATP} channel expression in mammalian cells utilizing recombinant baculovirus transduction” Receptors and Channels 8:99-111 (2002), the entire contents of each of which are incorporated herein in their entireties for teachings directed to construction of baculovirus vectors and transduction of mammalian cells with baculovirus vectors). Baculovirus mediated expression has been limited to in vitro systems of cultured mammalian cells but never before applied to whole tissues or organs or in the in vivo methods of the present invention.

The promoter of this invention can be a mammalian promoter or promoter operable in a mammalian cell (i.e., a mammalian-active promoter). The promoter sequence can be one that does not occur in nature, so long as it functions in a mammalian cell. By “promoter” is meant at least a minimal sequence sufficient to direct transcription of a nucleic acid. A “mammalian-active” promoter is one that is capable of directing transcription of a nucleic acid in a mammalian cell and includes promoters that are derived from the genome of a mammal or a virus (e.g., MMTV promoter, RSV LTR, SV40 early promoter, CMV IE promoter, adenovirus major late promoter or Hepatitis B virus promoter). The present invention also includes baculovirus vectors comprising sequences that allow either ribosome read through, cap-independent translation, or internal ribosome entry (e.g., an internal ribosome entry sequence or IRES). The source of these translation control sequences can be, but is not limited to, the picornaviruses polio and EMCV, the 5′ noncoding region of the human immunoglobulin heavy-chain binding protein, and a synthetic sequence of at least 15 bps corresponding in part to the Kozak consensus sequence for efficient translational initiation. The baculovirus vectors of this invention can further comprise matrix attachment regions and/or mammalian origins of replication and can also be manipulated to contain a ligand and/or other receptor-specific protein on the virus particle surface in order to facilitate entry into mammalian cells (e.g., by overexpression of a cell adhesion molecule such as VCAM in insect packaging cells) and/or target specific mammalian cell types. Protocols for and examples of all of these various modifications are standard in the art.

Other promoters included in the invention are promoters that render promoter-dependent nucleic acid expression controllable for cell-type specificity, cell differentiation stage specificity, tissue-specificity (e.g., liver-specific promoters) and/or inducible element specificity. Thus, the present invention includes constitutive promoters and/or promoters that are “inducible” by external or internal signals or agents that act as inducible elements (e.g., metallothionein, MMTV, and pENK promoters). In one such embodiment, such inducible elements can be located in the 5′ or 3′ regions of the nucleic acid.

An “inducible” promoter is a promoter that, (a) in the absence of an inducer or inducible element, does not direct expression, or directs low levels of expression, of a nucleic acid to which the inducible promoter is operably linked; or (b) exhibits a low level of expression in the presence of a regulating factor or element that, when removed, allows high-level expression from the promoter (e.g., the tet system). In the presence of an inducer, an inducible promoter directs transcription at an increased level.

In certain embodiments of this invention, the promoter is one that is specific for the cell type in which it is desired that the heterologous nucleic acid be expressed. For example, the skin is an organ that contains a variety of cells, such as keratinocytes, melanocytes, smooth muscle, fat and nerve cell processes. Thus, in one embodiment, a keratinocyte specific promoter can be used to express the heterologous nucleic acid in only keratinocytes despite the fact that other cell types have been transduced with the baculovirus vector. Various inducible promoters as described herein also can be used with a cell-specific promoter, allowing for expression of the heterologous nucleic acid only in a desired type of cell and/or only in the presence of an inducible agent or absence of an expression-inhibiting agent, thus allowing another level of specificity and control of expression of the heterologous nucleic acid.

By “operably linked” is meant that a nucleic acid and a regulatory sequence(s) (e.g., a promoter) are connected in such a way as to permit expression of the nucleic acid when the appropriate molecules (e.g., transcriptional activator proteins) are bound to the regulatory sequence(s).

The term “baculovirus” refers to any insect-specific virus and includes, but is not limited to examples such as Autographa californica NPV, Anagrapha falcifera NPV, Galleria mellonella MNPV, Rachiplusia ou MNPV, Bombyx mori SNPV, Heliothis zea NOB, Spodoptera exempta MNPV and Trichoplusia ni SNPV. It should be noted that the virus should be non-replicative in mammalian cells and the heterologous nucleic acid of this invention is introduced into cells via transduction and not by infection by the virus.

Potentially, any one or more heterologous nucleic acid sequences can be cloned into a baculovirus vector of this invention by inserting the heterologous nucleic acid sequence(s) into the genome of the baculovirus according to cloning methods well known in the art. The term “heterologous” as used herein refers to a nucleic acid that is not normally present in the genome of a baculovirus. The heterologous nucleic acid of this invention can be a nucleic acid normally found in the mammalian target and/or effector cell or the heterologous nucleic acid can be foreign to any nucleic acid present in the mammalian target and/or effector cell of this invention.

The heterologous nucleic acid of the present invention can encode a gene product that acts in effector cells to impart the cosmetic effect desired according to the methods of this invention. For example, the heterologous nucleic acid of this invention can include, but is not limited to, nucleic acid that is expressed to produce collagen proteins, elastin proteins, telomerase (e.g., to produce an effect of increased telomerase activity to conteract cell death and aging effects due to reduced telomerase activity), laminin proteins, fibronectin proteins, vitronectin receptor avb5, matrix metalloproteinases, cytokines (e.g., IL-1a, IL-1b, IL-8, IL-6, TNFa, IL-20, IL-18, IL-10, IL-12, IL-7, IL-15), cytokine receptors (e.g., IL-18-R, IL-10-R, IL-1-RI, IL-1-RU, IL-6-R, TNF-R, IL-20-R, IL-24-R, IL-4-R, IL-13-R, IL-17-R, Il-2-R), and fatty acid desaturases (e.g., to modify the aliphatic chain to increase/decrease water retention capabilities, greasiness). The target and/or effector cell that can be utilized for the production of the proteins encoded by the gene products of the present invention can be, but is not limited to, a keratinocyte.

The compositions of this invention comprising the baculovirus vector and a pharmaceutically acceptable carrier for delivery of the baculovirus onto, into and/or through the skin of a mammal can be in the form of a composition including, but not limited to, a cream, an aerosol, an ointment, an oil, a liquid, a lotion, a gel, a microneedle patch, a microadhesive, a transdermal patch and/or an adhesive strip. In certain embodiments, the composition can be administered via injection (i.e., through a needle or injection gun). The composition may be administered topically or parenterally as described herein, onto, into and/or through the skin of a mammal. The composition can also be administered in combination with other compositions of this invention and/or other compositions that impart a cosmetic effect in the skin of a mammal.

The mammal of this invention can be any mammal in which it is desired to produce the cosmetic effects imparted by the nucleic acids of this invention and can be, but is not limited to, a human. In some embodiments of this invention, the mammal can be a cat, dog, horse, or any companion animal.

The term “skin” refers to the epidermis, the dermis and the subcutaneous tissue. The epidermis is a fairly thin layer. Its thickness varies around the body, depending on the special needs of that area. For instance, the epidermis over the eyelids is particularly thin, while that over the palms and soles is very thick. The epidermis is itself made up of several layers. On the surface is the horny layer known as the stratum corneum. This layer is made up of dead cells, which are continuously being shed. The cells are shed off as small aggregates that can become larger and are then visible as scales. This is what happens in dandruff and when skin is deprived of moisture. Below the layer of dead cells are living cells comprising the stratum malpighi. The stratum malpighi can be divided into three differentiated sublayers. These are the stratum granulosum (the granular cell layer), the stratum spinosum (the prickle or spiny cell layer), and the stratum basale (the basal cell layer).

Situated at the base of the epidermis, 95% of the stratum basale is composed of basal keratinocytes. The major cell of the epidermis is the keratinocyte, which produces the highly resistant insoluble protein, keratin. Keratin provides part of the protective barrier function of the epidermis. Basal keratinocytes constantly divide through “mitosis” to form an identical replica occurring every few days to a week. Replicated basal kerotinocytes migrate upward to form layers of cells interconnected by “desmosomes” (prickles or spines) comprising the stratum spinosum, hence the name “prickle cell layer.” Also throughout the stratum spinosum are Langerhans cells, which are vital for their ability to provide cellular immunological responses.

Keratinocytes continually become transformed and lose their nuclei in the stratum granulosum or granular layer. Keratin becomes more evident in the stratum granulosum, hence the name “granular cells.” Epidermal lipids are also observed at this phase and are discharged into the intercellular spaces providing stratum corneum cohesion and barrier protection. As keratinocytes mature in the different layers of the epidermis and traverse the stratum basale outward to the stratum corneum, they change structure and chemistry. This entire physiochemical process of cellular proliferation represents the maturing of keratinocytes referred to as keratinization. As a result, the epidermis is constantly regenerated, ultimately constructing the highly defensive barrier known as the stratum corneum. This outer coat has evolved for protection from a hostile environment. This layer of dead cells, or comeocytes, is the only barrier of protection from the outside world. Corneocytes are anuclear cells that overlap, similar to roof tiles, and are designed to keep water and other chemicals from entering and to retain vital body water and fluids.

Melanocytes comprise the other 5% of the stratum basale. These cells synthesize melanin, which is transferred via melanosomes through cellular “dendrites” (arm-like extensions) to surrounding keratinocytes. There is approximately one melanocyte to every thirty-six keratinocytes. Variations in genetically determined skin pigment are related to the difference in melanosome structure produced by melanocytes. Melanin also provides protection from ultraviolet radiation, which is why melanocytes are stimulated upon exposure to the sun, resulting in darkening (i.e., tanning) of the skin.

The dermis is a much thicker layer than the epidermis. It is made up of a connective tissue framework, into which are embedded blood vessels, lymph vessels, nerves, several types of glands, hair and a variety of cells. The connective tissue of the dermis is made up predominantly of a protein called collagen. Collagen is the protein substance of the white fibers (collagenous fibers) of skin, tendon, bone, cartilage and all other connective tissue. Collagen is also known as processed procollagen molecule and triple helical processed procollagen monomeric molecule (for general reviews, see Kadler, Protein Profile, “Extracellular Matrix 1: fibril-forming collagens” 2:491-619 (1995); Avad et al., The Extracellular Matrix Facts Book, Academic Press, London, ISBN 0-12-068910-3 (1994), and references therein). Collagen is a major structural protein in animals where it occurs in the extracellular matrix (ECM) of connective tissues, mostly in the form of fibrils (also known as polymeric collagen). The collagen fibrils (polymeric collagen) are the major source of mechanical strength of connective tissues, providing a substratum for cell attachment and a scaffold for dynamic molecular interactions. The family of collagens includes complex multidomain proteins comprising three collagen alpha-chains wound into a triple helix. At least twenty genetically distinct collagen types have been described that are classified into subgroups on the basis of nucleic acid homology and function of the encoded protein. Fibril-forming collagens (types I, II, III, V and XI) are synthesized as soluble procollagens (also known as pro-alpha chains, procollagen alpha-chains and monomer chains) and are comprised of a C-propeptide, a Gly-X-Y repeat-containing region (which in the case of monomer chains of fibril-forming collagens comprises an uninterrupted collagen alpha-chain) and an N-propeptide. The pro-alpha chains trimerize to form unprocessed procollagen molecules (also known as monomeric procollagen molecules and trimerized pro-alpha chains), and assemble into fibrillar structures upon enzymatic cleavage of the N— and C-terminal propeptide domains (the N— and C-propeptides).

Collagen is used for the treatment of a variety of skin problems including wrinkles and scars. Elastin or elastic fibers are the other types of protein fibers in the dermis. The dermis also contains a complex system of blood and lymph vessels and a highly complicated nervous system.

The sub-cutaneous tissue is located below the dermis and is the fat storage bank of the skin. The amount of fat stored varies in different parts of the body. In some parts of the body, it is known as collucite.

In one embodiment of the present invention, the heterologous nucleic acid of this invention encodes a toxin that imparts a cosmetic effect to mammalian skin cells. For example, the toxin of this invention can be, but is not limited to, a bacterial toxin (e.g., botulinum toxin), a reptile toxin (e.g., snake venom) or an insect toxin (e.g., mellitin from bees). In a particular embodiment wherein the heterologous nucleic acid of this invention encodes botulinum, the toxin is the neurotoxin, botulinum toxin produced by Clostridium botulinum. The nucleic acid of this invention can encode botulinum toxin A, B, C₁, C₂, D, E, F, G, or any combinations thereof Furthermore, the nucleic acid encoding a toxin of this invention can be present in a baculovirus vector in multiple copies and/or in combination with one or more heterologous nucleic acids of this invention that encode proteins or nucleic acids that impart a cosmetic effect in the skin cells of a mammal.

Botulinum toxin causes a neuroparalytic illness in humans and animals referred to as botulism. The spores of Clostridium botulinum are found in soil and can grow in improperly sterilized and sealed food containers of home based canneries, which is the source of many cases of botulism. The effects of botulism typically appear 18 to 36 hours after eating the foodstuffs infected with Clostridium botulinum bacteria and/or spores. The botulinum toxin can apparently pass unattenuated through the lining of the gut and attack peripheral motor neurons. Symptoms of botulinum toxin intoxication can progress from difficulty in walking, in swallowing, and in speaking, to potential paralysis of the respiratory muscles and death.

Botulinum toxins have been used in clinical settings for the treatment of neuromuscular disorders characterized by hyperactive skeletal muscles. Botulinum toxin A has been approved by the U.S. Food and Drug Administration for the treatment of blepharospasm, strabismus and henifacial spasm. Non-serotype A botulinum toxin serotypes apparently have a lower potency and/or a shorter duration of activity as compared to botulinum toxin A. Clinical effects of peripheral intramuscular injection of botulinum toxin A are usually seen within one week of injection. The typical duration of symptomatic relief from a single intramuscular injection of botulinum toxin averages about three months.

Although all the botulinum toxin serotypes apparently inhibit release of the neurotransmitter acetylcholine at the neuromuscular junction, they do so by affecting different neurosecretory proteins and/or cleaving these proteins at different sites. For example, botulinum serotypes A and E both cleave the 25 kilodalton (kDa) synaptosomal associated protein (SNAP-25), but they target different amino acid sequences within this protein. Botulinum toxins B, D, F and G act on vesicle-associate protein (VAMP, also called synaptobrevin), with each serotype cleaving the protein at a different site. Finally, botulinum toxin serotype C₁ has been shown to cleave both syntaxin and SNAP-25. These differences in mechanism of action may affect the relative potency and/or duration of action of the various botulinum toxin serotypes.

Regardless of serotype, the molecular mechanism of toxin intoxication appears to be similar and to involve at least three steps or stages. In the first step of the process, the toxin binds to the presynaptic membrane of the target neuron through a specific interaction between the H chain and a cell surface receptor and the receptor is thought to be different for each serotype of botulinum toxin and for tetanus toxin. The carboxyl end segment of the H chain, H_{c}, appears to be important for targeting of the toxin to the cell surface.

In the second step, the toxin crosses the plasma membrane of the contacted cell. The toxin is first engulfed by the cell through receptor-mediated endocytosis, and an endosome containing the toxin is formed. The toxin then escapes from the endosome into the cytoplasm of the cell. This last step is thought to be mediated by the amino end segment of the H chain, Hₙ, which triggers a conformational change of the toxin in response to a pH of about 5.5 or lower. Endosomes are known to possess a proton pump that decreases intra-endosomal pH. The conformational shift exposes hydrophobic residues in the toxin, which permits the toxin to embed itself in the endosomal membrane. The toxin then translocates through the endosomal membrane into the cytosol.

The last step of the mechanism of botulinum toxin activity appears to involve reduction of the disulfide bond joining the H and L chain. The entire toxic activity of botulinum and tetanus toxins is contained in the L chain of the holotoxin. The L chain is a zinc (Zn++) endopeptidase that selectively cleaves proteins essential for recognition and docketing of neurotransmitter-containing vesicles with the cytoplasmic surface of the plasma membrane, and fusion of the vesicles with the plasma membrane. Tetanus neurotoxin and botulinum toxins B, D, F, and G cause degradation of synaptobrevin (also called vesicle-associated membrane protein (VAMP)), a synaptosomal membrane protein. Most of the VAMP present at the cytosolic surface of the synaptic vesicle is removed as a result of any one of these cleavage events. Each toxin specifically cleaves a different bond.

The molecular weight of the botulinum toxin protein, for all seven of the known botulinum toxin serotypes, is about 150 kDa. The botulinum toxins are released by Clostridium botulinum as complexes comprising the 150 kDa botulinum toxin protein molecule along with associated non-toxin proteins. Thus, the botulinum toxin A complex can be produced by Clostridiun botulinum as 900 kDa, 500 kDa and 300 kDa forms. Botulinum toxins B and C₁ are apparently produced as only a 500 kDa complex. BoNT/D is produced as both 300 kDa and 500 kDa complexes. Finally, botulinum toxins E and F are produced as only approximately 300 kDa complexes. The complexes (ie., molecular weight greater than about 150 kDa) are believed to contain a non-toxic hemagglutinin protein and a non-toxic nonhemagglutinin protein. These two non-toxic proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kDa molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation-induced release of the neurotransmitters, acetylcholine and norepinephrine, from primary cell cultures of brainstem tissue. Additionally, it has been reported that botulinum toxin inhibits the evoked release of both glycine and glutamate in primary cultures of spinal cord neurons and that in brain synaptosome preparations, botulinum toxin inhibits the release of each of the neurotransmitters acetylcholine, dopamine, norepinephrine, CGRP and glutamate.

Botulinum toxin A can be obtained by establishing and growing cultures of Clostridium botulinum in a fermenter and then harvesting and purifying the fermented synthesized as inactive single chain proteins that must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin B serotype, only cleave a portion of the toxin produced. The exact proportion of nicked to unnicked molecules depends on the length of incubation and the temperature of the culture. Therefore, a certain percentage of any preparation of, for example, obotulinum toxin B, is likely to be inactive, possibly accounting for the known significantly lower potency of botulinum toxin B as compared to botulinum toxin A. The presence of inactive botulinum toxin molecules in a clinical preparation will contribute to the overall protein load of the preparation, which has been linked to increased antigenicity, without contributing to its clinical efficacy. Additionally, it is known that botulinum toxin B has, upon intramuscular injection, a shorter duration of activity and is also less potent than botulinum toxin A at the same dose level.

In the present invention, the botulinum toxin protein does not need to be included in the composition delivered to the cells of a mammal, because, according to the methods provided herein, the botulinum protein is produced after expression of a heterologous nucleic acid encoding the botulinum toxin is delivered to a target skin cell of the mammal. Nucleic acid encoding botulinum toxin is produced according to methods well known in the art, such as cloning of the coding sequence from the genome of a Clostridium botulinum cell or by synthesis of the nucleic acid encoding botulinum toxin. The toxin produced in the target cell is secreted from the cell and is taken up by nerve cell termini that form a neuromuscular junction in the mammal in the same way that botulinum toxin is taken up by a nerve cell terminal when the protein is administered directly into the tissue of the mammal via injection. In the nerve cells, the botulinum toxin disrupts neuronal release of neurotransmitters, thereby imparting the cosmetic effect of reducing wrinkles and lines on the skin surface above and/or in the area of the effected muscle cells.

In an additional embodiment of this invention, a cosmetic effect similar to that imparted by botulinum toxin of reducing skin wrinkles can be obtained by delivering to the target skin cells of a mammal a baculovirus comprising a heterologous nucleic acid encoding a first protein that can disrupt neuronal release of neurotransmitter by inactivating a second protein involved in the neuronal release of transmitters, wherein the second protein can include, but is not limited to, a member of the family of soluble N-ethylmaleimide-sensitive factor attachment protein receptors known as SNAREs (such as 25 kDa synaptosomal-associated protein (SNAP-25), synaptobrevin and syntaxin), vesicle associated membrane protein 2/synaptobrevin, ATPase N-ethylmaleimide-sensitive factor (NSF), soluble NSAF attachment proteins (SNAP) and similar proteins that are involved in the release of neuronal release of neurotransmitters. Such an inhibitory protein of this invention can be identified by ordinary screening methods well known in the art to identify a protein (or peptide) that has a disruptive effect on the neurotransmitter releasing activity of these cellular proteins and once identified according to routine protocols, can be employed in the methods of this invention. As one example of such an inhibitory protein, a truncated SNARE protein can be produced and tested according to art-known protocols for inhibitory activity. Such a truncated SNARE protein can bind to the neurotransmitter-containing vesicle complex and prevent proper protein complex assembly, trafficking and/or docking of the neurotransmitter-containing vesicle. As another example, a protein that is identified according to standard methods to have a high affinity to the SNAP protein can be produced and this protein would bind to the SNAP protein and hinder the binding of other SNARE proteins with which it normally forms a complex.

In further embodiments of this invention, proteins and/or antisense nucleic acids are identified according to the methods of this invention that disrupt the ability of synaptic vesicles to package and retain a neurotransmitter. Thus, when the neurotransmitter level in the vesicle is reduced, even if the vesicle docks with the plasma membrane and the vesicle releases its components to the neuromuscular junction, there is no, or reduced, transmitter available. Similarly, proteins and/or antisense nucleic acids are identified according to the methods provided herein that reduce acetylcholine receptor levels in muscle (i.e., no affector) or that increase the acetylcholinesterase levels (i.e., to break down the neurotransmitter levels) to impart the same effect.

In another embodiment of the present invention, it is contemplated that the heterologous nucleic acid of this invention encodes an antisense sequence that can hybridize to and form a complex with a nucleotide sequence that encodes a protein or peptide that is involved in neurotransmitter release activity of neuronal cells. By hybridizing to a nucleotide sequence encoding a cellular protein involved in neurotransmitter release, the antisense nucleic acid can inhibit production of the cellular protein, thereby disrupting neuronal neurotransmitter release. The net result of this action would be to relax muscle tissue and reduce the appearance of wrinkles, thereby imparting a cosmetic effect to the skin of the mammal of this invention.

For example, in one embodiment, the heterologous nucleic acid of this invention can encode a sequence complementary to the coding sequence for the cellular protein, syntaxin, which is a protein that forms the complex of attachment proteins that permit regulated release of neurotransmitter from nerve terminals. The formation of this complex results in the inhibition of expression of the nucleotide sequence encoding syntaxin and thus inhibits production of syntaxin in the mammalian cell. The effect of this antisense action is to inactivate syntaxin, thereby producing a cosmetic effect similar to that produced in a muscle cell by administration of botulinum toxin C₁, which is known to impart a muscle-relaxing and thereby a wrinkle-reducing effect by cleaving syntaxin.

In other examples of the use of an antisense nucleic acid to impart a cosmetic effect according to the methods of this invention, the heterologous nucleic acid of this invention can encode an antisense nucleic acid sequence that is complementary to, and forms a complex in a cell with, a nucleic acid encoding tyrosinase, thereby inhibiting production of tyrosinase in the cell, which is involved in the production of melanin. The cosmetic effect imparted by inhibition of the production of melanin as a result of inhibition of the production of tyrosinase is decreased pigmentation of the skin of a mammal which can be employed, for example, to lighten skin color and to reduce the appearance of birthmarks and other pigmented areas on the skin.

Thus, in various embodiments, the heterologous nucleic acid of this invention can encode an antisense nucleic acid sequence. An “antisense” nucleic acid is a nucleic acid molecule (i.e., DNA or RNA) that is complementary (i.e., able to hybridize in vivo or under stringent in vitro conditions) to all or a portion of a nucleic acid (e.g., a gene, a cDNA and/or mRNA) that encodes a polypeptide to be targeted for inhibited or reduced production by the action of the antisense nucleic acid. If desired, conventional methods can be used to produce an antisense nucleic acid that contains desirable modifications. For example, a phosphorothioate oligonucleotide can be used as the antisense nucleic acid to inhibit degradation of the antisense oligonucleotide by nucleases in vivo. Where the antisense nucleic acid is complementary to a portion of the nucleic acid encoding the polypeptide to be targeted, the antisense nucleic acid should hybridize close enough to the 5′ end of the nucleic acid encoding the polypeptide such that it inhibits translation of a functional polypeptide. Typically, this means that the antisense nucleic acid should be complementary to a sequence that is within the 5′ half or third of the nucleic acid to which it hybridizes.

An antisense nucleic acid of this invention can also encode a catalytic RNA (i.e., a ribozyme) that inhibits expression of a target nucleic acid in a cell by hydrolyzing an mRNA encoding the targeted gene product. An antisense nucleic acid of this invention can be produced and tested according to protocols routine in the art for antisense technology.

Additionally, hammerhead ribozyme RNA can be used to inhibit expression of a target gene by selective cleavage of precursor mRNA (Haseloff and Gerlach 1988. Simple RNA enzymes with new and highly specific endoribonuclease activities. Nature 334:585-591; Larsson et al. 1994. Reduced beta 2-microglobulin mRNA levels in transgenic mice expressing a designed hammerhead ribozyme. Nucleic Acids Res. 22:2242-2248. The entire contents of these references are incorporated herein by reference for the teachings of ribozyme RNA.)

In further embodiments, the heterologous nucleic acid of this invention can encode a “competing protein” (i.e., a “functional knockout protein”) that reduces the activity of a targeted protein within a cell. For example, the competing protein can be a truncated form of the targeted protein wherein the competing protein has sufficient elements to allow it to function like the native targeted protein, thereby allowing the competing protein to couple with normal cellular proteins and prevent fully functional protein complexes that would otherwise include the targeted protein. For example, a truncated SNARE protein can bind to a neurotransmitter-containing vesicle and prevent normal protein/vesicle complex formation, thereby inhibiting or disrupting trafficking and/or docking of the neurotransmitter-containing vesicle. In another example, a protein can be produced that has a high binding affinity for the SNAP protein, thereby complexing with the SNAP protein and preventing normal complex formation with other SNARE protein.

Cells and tissues of the present invention that can function as the target cells and tissues and/or effector cells and tissues in the methods of this invention include, but are not limited to, cutaneous nerves, subdermal fatty tissue, Schwann cells, Merkel cells, Pacinian corpuscles, Golgi-Mazzoni, Krause end bulb, Meissner corpuscles, Ruffini endings, free nerve endings, mast cells, nutritive vessels, vascular structures (e.g., arteriovenous anastomoses), basal cell layers connecting the epidermis with the dermis, granular layer flattened cells containing numerous keratohyaline granules, cells containing Odland bodies, moisture prickle cell layers containing Langerhans cells, keratinocytes, horny layer containing dead and keratinized skin cells, hair follicle cells, melanocytes, sebaceous gland cells (e.g., of meibomian glands, Tyson's glands, etc.), erector pili muscle cells, sweat gland cells, adipocytes, hard nail plate, nail matrix tissue, hyponychonium and subcutaneous muscle cells.

The present invention provides for in vivo delivery of the compositions of this invention comprising baculoviruses for cosmetic uses. This novel use of the cosmetic compositions of this invention is an improvement over existing methods of delivery, which rely on uncomfortable approaches such as injection, inhalation or ingestion, among others. The compositions of this invention can include an excipient to provide the effective application of an appropriately diluted preparation of the recombinant virus. Such excipients are well known in the art of topical and parenteral application and can include, but are not limited to, water, glycerin, stearates, palmitates, petroleum derivatives, ethyl alcohol, methyl paraben and propyl paraben. The compositions of this invention can be provided in a container and/or be associated with a mechanical device compatible with the application of the cosmetic composition. Such containers and devices are well known in the art of topical and parenteral applications and include, for example, squeezable tubes, roll-on applicators and a variety of dermal patches that are well-described in the art.

The compositions of this invention can be applied directly to the skin of a mammal via an excipient and/or mechanical device as described herein. The heterologous nucleic acid in the composition of this invention can be expressed either directly in an effector cell or in a target cell from which it is secreted and then transported to an effector cell. As used herein, “target cell” means a cell transduced by the baculovirus of the present invention and in which the heterologous nucleic acid is expressed to produce a cosmetic material (e.g., a gene product or nucleic acid that imparts a beneficial cosmetic effect.) As also used herein an “effector cell” is a cell that binds and/or internalizes the cosmetic material, resulting in production of the beneficial cosmetic effect by the cosmetic material. In certain embodiments, the target cell and effector cell can be the same cell.

The amount of baculovirus applied and the dose of the nucleic acid expressed can be directly correlated (Pfohl et al., Receptors and Channels 8(2):99-111 (2002), the entire contents of which is incorporated herein in its entirety for its teaching of protocols for correlating the amount of virus applied to cells and the resulting level of expression of a heterologous nucleic acid.) Thus, the dose of the heterologous nucleic acid can be controlled for maximal benefit. The baculovirus can also be applied at one time or at various time intervals (e.g., hourly, daily, weekly, monthly, etc.) that will also increase the amount of nucleic acid delivered over a period of time, depending on the desired cosmetic outcome.

The amount of virus to be administered and the frequency and mode of administration according to the methods of this invention are optimized according to standard protocols and as determined by the particular target and effector cells, the type of promoter used, the heterologous nucleic acid to be expressed and the particular cosmetic effect to be achieved. In particular embodiments of the invention, the virus can be administered at a multiplicity of infection (moi) of about 0.1 to about 1.0 and can be administered for example at an moi of about 5 to 100 and/or can be administered at an moi of about 10 to 50. Identification of the optimal moi, as well as the mode and frequency of administration is determined according to methods standard in the art.

In particular embodiments, the present invention provides methods of delivering a heterologous nucleic acid to a mammalian target cell, comprising contacting the skin of the mammal with a composition comprising a baculovirus comprising the heterologous nucleic acid and a pharmaceutically acceptable carrier for delivery to a target cell, under conditions whereby the baculovirus vector is introduced into the target cell and the heterologous nucleic acid is expressed. Furthermore, the present invention provides methods of delivering a heterologous nucleic acid to a mammalian target cell, comprising contacting the epidermis of the mammal with a composition of the present invention whereby the baculovirus vector is transported into, onto and/or through the epidermis and introduced into the target cell. Additionally, the present invention provides methods of delivering a heterologous nucleic acid to a mammalian target cell, comprising contacting the dermis of the mammal with a composition of the present invention whereby the baculovirus vector is transported into, onto and/or through the dermis and introduced into the target cell. The present invention also provides methods of delivering a heterologous nucleic acid to a mammalian target cell through the subcutaneous tissue of a mammal, comprising contacting the subcutaneous tissue of the mammal with a composition of the present invention under conditions whereby the baculovirus vector is transported into and/or through the subcutaneous tissue and introduced into the target cell.

Additionally, the present invention provides a method of expressing a heterologous nucleic acid in a target cell in a mammal, comprising introducing into the target cell a baculovirus vector of the present invention.

The present invention also provides various methods of maintaining skin health and improving or enhancing the appearance of skin. In particular, the present invention provides methods of reducing the appearance of a scar, reducing the incidence of acne, reducing the incidence of dry skin, reducing the signs of aging skin, reducing oil secretion, reducing skin wrinkles, reducing the appearance of pigmented areas on the skin such as birthmarks and age spots, improving elasticity of the skin, tanning (i.e., darkening) the skin, lightening the skin, reducing the appearance of a tattoo on the skin, creating a temporary tattoo on the skin and reducing perspiration.

The present invention further provides methods of improving nail health and appearance of finger nails and toe nails in a mammal, for example, by increasing hardness of the nails, increasing the shine of the nails, producing an even color throughout the nail, removing pigmentation from the nails, creating color in the nails and improving the condition of the hyponychium (skin under the nail), thereby enhancing the appearance and health of the nail. The heterologous nucleic acids employed in methods directed to improving nail health and appearance can be the same heterologous nucleic acids employed in the methods of this invention for improving skin health and improving or enhancing the appearance of the skin.

In further embodiments, the present invention provides methods of modifying hair growth and hair appearance in a mammal. As some examples, the present invention provides methods of inhibiting hair growth, inhibiting oil secretion to reduce hair oiliness, removing color from the hair, adding color to the hair, changing the shade of color of the hair, changing the texture of the hair and changing the appearance of the hair.

As an example, the present invention provides the use of the compositions of this invention in a method of reducing skin wrinkles in a mammal, comprising contacting a composition of the invention with the skin of a mammal under conditions whereby the baculovirus vector is delivered to a target cell and the heterologous nucleic acid is expressed to produce botulinum toxin and/or another protein and/or antisense nucleic acid that acts as described herein to reduce skin wrinkles and lines, thereby reducing skin wrinkles in the mammal. In one embodiment, for example, as described above, the botulinum toxin is produced in a target skin cell, secreted by the skin cell and taken up by a nerve cell terminal that forms a neuromuscular junction, (e.g., a presynaptic terminal), where it acts to produce the cosmetic effect of relaxing muscle tissue and reducing the appearance of wrinkles and lines on the skin of the mammal.

The present invention also provides methods of tanning the skin of a mammal (e.g., a human), comprising contacting a composition of the present invention with the skin of the mammal, wherein the baculovirus comprises a heterologous nucleic acid encoding a melanocyte modulatory protein and/or a keratinocyte modulatory protein under conditions whereby the baculovirus vector is delivered to a target cell, the heterologous nucleic acid is expressed to produce the melanocyte modulatory protein and/or the keratinocyte modulatory protein and the protein or proteins are delivered to melanocytes and/or keratinocytes, thereby tanning the skin of the mammal.

The methods utilized to tan the skin of a mammal provide a cosmetic effect by stimulating melanogenesis, resulting in an increase in the synthesis and distribution of appropriate melanins, thereby producing a darker pigmentation, or tanning of the cells. Additionally, these methods result in an increase in the ability of keratinocytes to take up, accumulate or decrease the degradation of melanins by the keratinocyte.

The modulatory proteins for tanning of skin cells by activity of melanocytes include, but are not limited to, tyrosinase, tyrosinase-related protein-1, tyrosinase-related protein-2, melanocortin-1 receptor, pro-opiomelanocortin (POMC), melanocortin peptides such as alpha-MSH, ocular albinism 1 (OA1), MART-1 (melan-A) and/or pmel17 (gp100). The modulatory proteins for tanning of skin cells by activity of keratinocytes include, but are not limited to, pro-opiomelanocortin (POMC), melanocortin peptides such as alpha-MSH and/or protease-activated receptor 2 (PAR-2).

The methods of this invention can also be used to reduce the appearance of pigmented areas in the skin (e.g., liver spots, lentigines, birthmarks, freckles), as well as to cosmetically improve and/or enhance the skin tone of various body parts such as lips, hands and nails. For example, in these embodiments of the methods of the present invention, a heterologous nucleic acid can be employed that have the reverse effect of the methods described herein for tanning skin. In a particular example, heterologous nucleic acid encoding an antisense nucleic acid and/or competing protein that has the effect of inhibiting or reducing melanin production can be introduced into a mammalian cell, thereby lightening the skin and/or reducing the appearance of pigmented areas of the skin.

The present invention also provides methods of reducing the incidence of acne in the skin of a mammal (e.g., a human), comprising contacting a composition of this invention with the skin of the mammal, wherein the heterologous nucleic acid encodes androgen receptors and/or 5α reductase under conditions whereby the baculovirus vector is delivered to a target cell, the heterologous nucleic acid is expressed to produce the androgen receptors and/or 5α reductase and these proteins are delivered to cells in sebaceous glands to reduce the ability of the sebaceous glands to produce sebum, thereby reducing the incidence of acne. The effector cells of this method can be, for example, the sebaceous gland cells of the face, forehead, back and chest of a mammal.

In another aspect, the present invention provides methods of diminishing the appearance of a scar in the skin of a mammal, comprising contacting a composition comprising a baculovirus vector and a pharmaceutically acceptable carrier with the skin of the mammal, wherein the heterologous nucleic acid encodes a protein and/or antisense nucleic acid, as described herein that can reduce the color of the skin of the scar and/or alter the activity or amount of collagen, elastin, TGF and/or specific cytokines in the skin cells of the scar, under conditions whereby the baculovirus vector is delivered to a target cell, the heterologous nucleic acid is expressed to produce the protein and/or antisense nucleic acid described herein and the protein and/or antisense nucleic acid is delivered to and taken up by an effector cell, where it acts to diminishing the appearance of a scar in the skin of a mammal.

In another aspect, the present invention provides methods of lightening, darkening or changing the hair color of a mammal, comprising contacting a composition a composition of this invention with the skin of the mammal, wherein the heterologous nucleic acid encodes a protein and/or antisense nucleic acid that modulates the expression of melanin in melanocytes in hair follicles under conditions whereby the baculovirus vector is delivered to a target cell, the heterologous nucleic acid is expressed to produce the protein and/or antisense nucleic acid described herein and the protein and/or antisense nucleic acid is delivered to an effector cell, which is a melanocyte in a hair follicle, where it modulates melanin production, thereby lightening, darkening or changing the shade of color of the hair of a mammal.

The present invention further provides methods of removing hair and/or slowing the growth of hair in the skin of a mammal comprising contacting a composition of this invention with the skin of the mammal at the site of the hair to be removed, wherein the heterologous nucleic acid encodes a protein and/or an antisense nucleic acid which acts to 1) alter hair follicle cycling, 2) to induce apoptosis of the cells of the hair follicle and/or associated cells, 3) reduce androgen responsiveness, and/or 4) reduce hair follicle morphogenesis, under conditions whereby the baculovirus vector is delivered to a target cell, the heterologous nucleic acid is expressed to produce the protein and/or antisense nucleic acid of this method and the protein and/or antisense nucleic acid is delivered to an effector cell where it imparts one or more of the effects described for this method, thereby removing hair and/or slowing the growth of hair in the skin of a mammal.

The effector cells of this method include, but are not limited to, cells of the hair follicle, such as follicular epithelial cells and hair germ cells located in the bulge, outer root sheath or hair matrix/precortex regions of the hair follicle.

The heterologous nucleic acid of the methods of removing and/or slowing growth of hair can encode proteins such as, for example, ornithine decarboxylase, antizyme, β-catenin, LEF1, FGF/FGFR2-IIIB, TGFβ2, MSX1, MSX2, EDA/EDAR, NOGGIN, Delta-1/Notch1, PDGF-A, SHH, ACTbA/FS, HGF/MET, SOX18, WNT, ETS2, BMPs, BMP4, MOVO1, HOXC13, WHN and/or proteins that induce apoptosis, including, but not limited to, tumor necrosis factor-related apoptosis-inducing ligand (TRAIL), Bcl-2 proteins (e.g., Bax, Bid, Bcl-2, and Bcl-X_{L}), serine proteases, TNF-α, Fas ligand (FasL, known as CD95L), cysteine-aspartate proteases (e.g., caspase-9, caspase-3, caspase-7), cytochrome c and mitochondria chloride channel (VDAC).

Thus, the present invention also provides methods of inducing apoptosis in a cell in the skin of a mammal to achieve a cosmetic purpose as described herein, comprising contacting a composition of this invention with the skin of a mammal, wherein the heterologous nucleic acid encodes a one or more of the proteins and/or antisense nucleic acids listed herein and/or known in the art for induction of apoptosis, under conditions whereby the baculovirus vector is delivered to a target cell, the heterologous nucleic acid is expressed to produce the protein and/or antisense nucleic acid that induces apoptosis for a cosmetic effect and the protein and/or nucleic acid is delivered to an effector cell in which apoptosis is induced, resulting in the desired cosmetic effect in the skin of the mammal.

The present invention further provides a method of stimulating hair growth in a hair follicle in the skin of a mammal, comprising contacting the composition of this invention with the hair follicle in the skin of the mammal, wherein the heterologous nucleic acid encodes one or more proteins and/or antisense nucleic acids that stimulate hair growth in a hair follicle, under conditions whereby the baculovirus vector is delivered to a target cell, the heterologous nucleic acid is expressed to produce the protein and/or antisense nucleic acid that stimulates hair growth and the protein and/or antisense nucleic acid is delivered to an effector cell in the hair follicle, where the protein and/or antisense nucleic acid imparts its effect of stimulating hair growth. The hair follicle of this method can be a normally functioning hair follicle, or a non-functioning hair follicle, such as in a balding condition. For the latter condition, the protein and/or antisense nucleic acid delivered to the effector cell can act by increasing hormonal receptors on the hair follicle cells of males, thereby stimulating hair growth from these follicles.

In a further embodiment, the present invention provides a method of reducing a fat deposit in the skin of a mammal to achieve a cosmetic effect, comprising contacting a composition of this invention with the skin of the mammal at the site of the fat deposit to be reduced, wherein the heterologous nucleic acid encodes a protein which acts to reduce a fat deposit, including, but not limited to, uncoupling protein-1 (UCP-1), uncoupling protein-2 (UCP-2), uncoupling protein-3 (UCP-3), β-andrenergic receptor 3 and/or proteins described herein or which are known in the art for the activity of inducing apoptosis, under conditions whereby the baculovirus vector is delivered to a target cell, the heterologous nucleic acid is expressed to produce the protein and/or antisense nucleic acid of this method and the protein and/or antisense nucleic acid is delivered to an effector cell, such as an adipocyte near the skin surface, where the protein and/or antisense nucleic acid acts to reduce the fat deposit in the skin of the mammal, thereby producing thereby reducing a fat deposit in the skin of the mammal to achieve a cosmetic effect.

The effector cells of this method can be, but not limited to, brown adipocytes, white adipocytes and adipocytes that are in close proximity to the skin surface. The adipocytes can be located in the face, head and neck regions, as well as in other body regions of the mammal where it is desirable to reduce a fat deposit to achieve a cosmetic effect.

The present invention also provides a method of reducing perspiration in the skin of a mammal, comprising contacting a composition of this invention with the skin of the mammal, wherein the heterologous nucleic acid encodes a protein and/or antisense nucleic acid that acts to reduce perspiration in the skin of a mammal (e.g., sodium bicarbonate and/or other proteins or antisense nucleic acids that modulate Na-bicarbonate exchange proteins, Na/H transporters, chloride channels and Na/K exchangers), under conditions whereby the baculovirus vector is delivered to a target cell, the heterologous nucleic acid is expressed to produce the protein and/or antisense nucleic acid of this method and the protein and/or antisense nucleic acid is delivered to an effector cell, such as a neuronal cell, where it acts to reduce perspiration by neuronal input or by the ability of the sweat glands to secret ultrafiltrate. The method of reducing perspiration can be used to can reduce perspiration in the armpits, on the feet, on the hands, etc. The effector cells of the method of reducing perspiration can include, but are not limited to, sweat gland cells and cells involved in the neuronal release of neurotransmitters involved in the production and release of sweat.

The present invention also provides methods of delivering vitamin E to a cell in the skin of a mammal to impart a cosmetic effect, comprising contacting a composition of this invention with the skin of a mammal, wherein the heterologous nucleic acid encodes vitamin E under conditions whereby the baculovirus vector is delivered to a target cell, the heterologous nucleic acid is expressed to produce vitamin E and the vitamin E is delivered to an effector cell that metabolizes vitamin E and imparts a cosmetic effect.

Additionally, the present invention provides methods of reducing signs of aging in a mammal comprising contacting a composition comprising a baculovirus vector and a pharmaceutically acceptable carrier with the skin of a mammal, wherein the heterologous nucleic acid encodes a protein and/or antisense nucleic acid that reduces the signs of aging by, for example, increasing moisture content of the skin, increasing oil production in dry skin, decreasing oil production in oily skin, improving elasticity and/or tone of the skin and the like, under conditions whereby the baculovirus vector is delivered to a target cell, the heterologous nucleic acid is expressed to produce the protein and/or antisense nucleic acid of this method and the protein and/or antisense nucleic acid is delivered to an effector cell, such as a keratinocyte, where it acts to reduce signs of aging in the skin of a mammal.

The present invention further provides methods of reducing the appearance of a tattoo in the skin of a mammal generated according to the methods of this invention, comprising contacting a composition comprising a baculovirus vector and a pharmaceutically acceptable carrier with the skin of a mammal, wherein the heterologous nucleic acid encodes a protein and/or antisense nucleic acid that reduces or inhibits melanin production that resulted in the pigmentation of the tattoo, under conditions whereby the baculovirus vector is delivered to a target cell, the heterologous nucleic acid is expressed to produce the protein and/or antisense nucleic acid of this method and the protein and/or nucleic acid is delivered to an effector cell, where it acts to reduce or inhibit melanin production that produced the pigmentation of the tattoo, thereby removing color from a tattoo on the skin of a mammal.

Further provided herein is a method of producing a temporary tattoo in the skin of a mammal, comprising contacting a composition of this invention with the skin of a mammal in a pattern or array that will produce a tattoo-like design or image in the skin of the mammal, under conditions whereby the heterologous nucleic acid is expressed in a target cell to produce the protein and/or antisense nucleic acid of this method which would produce, and/or reduce the production of, a pigment in the cells marked for pigment production or pigment reduction according to the pattern or array desired and the protein and/or antisense nucleic acid is transported to an effector cell, where it acts to produce pigmentation and/or to reduce pigmentation in cells of the skin in the pattern or array, thereby produce a tattoo-like design or image on the skin of the mammal. For example, the heterologous nucleic acid employed in this method can encode a fluorescent protein that glows when exposed to the appropriate wavelength of light. In another embodiment, the heterologous nucleic acid can encode a colored protein that gives a skin cell a particular color (e.g., henna). In further embodiments, the heterologous nucleic acid of this invention can encode specific melanins that produce different pigments, such as eumelanin to produce a brown to black pigment and phaeomelanin to produce a yellow-red pigment. These various proteins can be used in any combination to produce a variety of designs and arrays in the skin cells.

The following examples are set forth to illustrate the present invention, and are not to be construed as limiting thereof.

### EXAMPLE I

### Animal Studies

To demonstrate expression of a heterologous nucleic acid and subsequent production of a functional gene product in skin cells in a mammal upon transduction of the skin cells by a baculovirus vector comprising a heterologous nucleic acid according to the methods of this invention, the following experiments can be conducted.

In one example, the heterologous nucleic acid carried by the baculovirus vector can be a reporter gene such as a green fluorescent protein (GFP) gene, a chloramphenicol acetyltransferase (CAT) gene, an alkaline phosphatase gene or a β-galactosidase gene, to name only a few examples. A composition of this invention, comprising a baculovirus vector comprising a reporter gene is contacted with the skin of a test animal according to the modes of administration of the compositions of this invention as described herein in the dosage ranges and for various time intervals as described herein. Skin cells from the test animal can be removed and analyzed for the presence of the specific reporter gene, according to well-defined methods. For example, skin cells expressing nucleic acid encoding β-galactosidase or alkaline phosphatase can be reacted with the respective substrates for these enzymes and the presence of a color characteristic of that particular enyzymatic activity can be detected, thereby demonstrating expression in the skin cells of the heterologous nucleic acid delivered by the baculovirus vector. Alternatively, the presence of the heterologous nucleic acid in skin cells can be demonstrated by art-known protocols for detecting specific nucleic acids, such as polymerase chain reaction (PCR), Southern blotting, Northern blotting, in situ hybridization, etc. The presence in a transduced skin cell of a protein produced by expression of a heterologous nucleic acid can be demonstrated by standard methods such as, for example, Western blotting and immunohistochemical staining.

For example, a proof of principle of the present invention can be achieved using reporter genes whose activity and expression can be followed visually or by immunohistochemistry. Reporter genes that can be used include beta-galactosidase (Choate and Khavari 1997. Sustainability of keratinocyte gene transfer and cell survival in vivo. Hum. Gene Ther. 8, 895-901)

The efficacy of transduction can first be tested by subcutaneous injections in nude mice of a preparation containing the baculovirus vector harboring the reporter gene under a keratinocyte specific promoter. The activity of the reporter is then followed by known methods. This approach is based on several published reports. For example, Lu and coworkers showed that the subcutaneous administration of an adenoviral vector containing the luciferase reporter gene induced a strong expression of the transgene in dermal cells (Lu and Federoff 1995. Herpes simplex virus type 1 amplicon vectors with glucocorticoid-inducible gene expression. Hum. Gene Ther. 6:419-428). Another example can be found in the reference by Setoguchi et al. (Ex vivo and in vivo gene transfer to the skin using replication-deficient recombinant adenovirus vectors. J. Invest. Derinatol. 102:415-421 (1994)).

The next step is to test the efficacy of transduction in human keratinocytes. One approach is the baculovirus mediated transduction of beta-galactosidase, green fluorescent protein or luciferase in human cultured HeLa keratinocytes. The transducing cells are then implanted subcutaneously in nude mice and nucleic acid expression is followed using known methods. This experimental design is based on the studies by Descamps et al. (Keratinocytes as a target for gene therapy. Sustained production of erythropoietin in mice by human keratinocytes transduced with an adeno-associated virus vector. Arch-Dermatol. 132:1207-1211 (1996)) in which the implantation in nude mice of HeLa keratinocytes transduced by the adeno-associated virus harboring the erythropoetin cDNA induced a high level and long-term increase in hematocrit.

Another approach is the topical application of the baculovirus vector engineered to express the reporter gene under control of either a keratinocyte specific or a CMV promoter. Lu et al. used this approach to show expression of beta-galactosidase under CMV promoter control in adenovirus (Lu et al. 1997. Topical application of viral vectors for epidermal gene transfer. J. Invest. Dermatol. 108:195-199). All of the references cited in this section are incorporated herein in their entireties for the teachings directed to method to demonstrate expression of heterologous nucleic acid in cells transduced with a viral vector.

### EXAMPLE II

### Anti-Wrinkle Treatment

In one embodiment of the methods of the present invention an anti-wrinkle cosmetic effect can be obtained. The nucleic acid encoding any form of botulinum toxin is cloned, along with a cell-specific keratinocyte-specific promoter into the genome of a baculovirus. The baculovirus construct is appropriately diluted and formulated into a composition in an excipient containing appropriate inert ingredients compatible with baculovirus survival and delivery onto, into and/or through the skin as described herein. The composition is applied to the skin at the site of a wrinkle by a lotion or other delivery vehicle or carrier comprising a measurable amount of the composition effective in reducing the appearance of wrinkles on the skin at the site of application of the baculovirus composition. The nucleic acid encoding the toxin is expressed upon entry of the virus into a keratinocyte to produce the toxin. The toxin is secreted and diffused to the subcutaneous nerve cells where it is taken up and exerts its effect of inhibiting neuronally regulated release of neurotransmitters, thereby resulting in relaxation of the muscle tissue associated with the effected nerve cells. The subsequent relaxation of the muscle reduces the appearance of the wrinkles, thus yielding the desired cosmetic effect. The heterolgous nucleic acid can also be delivered to other skin cells via the baculovirus vector and expressed in close proximity to skeletal muscle cells. The amount of baculovirus vector administered to the skin can be in the range of about 10⁶ to more than 10⁸ plaque forming units (pfu) per milliliter of the composition in which the baculovirus is delivered. The time of exposure of the skin to the baculovirus-containing composition can be from, for example, two hours to overnight and the composition can be administered repeatedly as necessary to achieve the desired cosmetic effect as determined, for example, visually. In particular embodiments of this invention, where more than one cosmetic effect is desired to be achieved simultaneously or sequentially, a first composition comprising a first baculovirus for imparting a first cosmetic effect can be administered alternately, in a sequence, and/or simultaneously with a second (and/or third) composition comprising a second (and/or third) baculovirus for imparting a second (and/or third) cosmetic effect. Alternatively, the first and second baculoviruses can be in the same composition and administered as such simultaneously.

### EXAMPLE III

### Age Spot Elimination and Tanning of Skin

In another embodiment of the present invention the suppression of unsightly “age spots” is a desirable cosmetic effect that can be achieved according to the methods provided herein. In this example, a heterologous nucleic acid encoding a protein and/or antisense nucleic acid that interferes with melanin production by melanocytes, either specifically or by inducing melanocyte apoptosis, is cloned, along with a melanocyte specific promoter into the genome of Autographa californica. The construct is appropriately diluted and formulated into a composition in an excipient containing appropriate inert ingredients compatible with baculovirus survival and delivery onto, into and/or through the skin as described herein. The composition is applied to the skin by using a lotion or other delivery vehicle that delivers a measurable amount of the composition effective in imparting the cosmetic effect of reducing the appearance of age spots. The nucleic acid is expressed upon entry of the baculovirus into the melanocyte to produce the protein and/or antisense nucleic acid that interferes with melanin production and the desired effect of reducing the appearance of the age spot is achieved by suppression of melanin production.

In another embodiment of this invention, in methods of tanning the skin of a mammal, a heterologous nucleic acid is delivered, the expression of which results in the production of a protein and/or antisense nucleic acid that promotes melanin dispersal and/or melanin production to produce a tanned appearance of the skin in the presence and/or absence of sunlight. Examples of the proteins that can be used in this method include, but are not limited to, tyrosinase, tyrosinase-related protein-1, tyrosinase-related protein-2, melanocortin-1 receptor, pro-opiomelanocortin (POMC), melanocortin peptides such as alpha-MSH, ocular albinism 1 (OA1), MART-1 (melan-A), pmel17 (gp100), pro-opiomelanocortin (POMC) and/or melanocortin peptides such as alpha-MSH protease-activated receptor 2 (PAR-2). The amount of baculovirus vector administered to the skin can be in the range of about 10⁶ to more than 10⁸ plaque forming units (pfu) per milliliter of the composition in which the baculovirus is delivered. The time of exposure of the skin to the baculovirus-containing composition can be from, for example, two hours to overnight and the composition can be administered repeatedly as necessary to achieve the desired cosmetic effect as determined, for example, visually.

### EXAMPLE IV

### Fat Reducing Cream

In yet another embodiment of the present invention the elimination of subcutaneous fat deposits in skin cells of a mammal is a desirable cosmetic effect that can be achieved according the methods of this invention. Specifically, a heterologous nucleic acid encoding a protein and/or antisense nucleic acid that induces an increase of metabolism in adipocytes, or induces programmed cell death of adipocytes is cloned, along with an adipocyte specific promoter into the genome of a baculovirus. The construct is appropriately diluted and formulated into a composition in an excipient containing appropriate inert ingredients compatible with a topical application via a delivery vehicle such as a microneedle patch. The patch contains an amount effective in producing the cosmetic effect of reducing a fat deposit in the desired region of the skin and is applied in the desired region. Upon entry of the baculovirus into the adipocyte, the nucleic acid is expressed to produce the protein and/or antisense nucleic acid that effectively induces the adipocyte to decrease its fat deposition by increased metabolism or by programmed cell death. The desired cosmetic effect is achieved by a resulting reduction of the amount in fat deposits and/or the number of adipocytes. The amount of baculovirus vector administered to the skin can be in the range of about 10⁶ to more than 10⁸ plaque forming units (pfu) per milliliter of the composition in which the baculovirus is delivered. The time of exposure of the skin to the baculovirus-containing composition can be from, for example, two hours to overnight and the composition can be administered repeatedly as necessary to achieve the desired cosmetic effect as determined, for example, visually.

The foregoing is considered as illustrative only of the principles of the invention. Further, since numerous modifications and changes will readily occur to those skilled in the art, it is not desired to limit the invention to the exact construction and operation shown and described herein. Therefore, accordingly, all suitable modifications and equivalents fall within the scope of the invention.

All publications, patent applications, patents, and other references mentioned herein are incorporated by reference in their entirety for the teachings described in the paragraph and/or sentence wherein the citation is made.

What is claimed is:

1. A composition comprising a baculovirus vector and a pharmaceutically acceptable carrier for delivery of said baculovirus vector onto, into or through the skin of a mammal, wherein the baculovirus vector comprises a promoter operable in a mammalian cell and a heterologous nucleic acid.

2. The composition of claim 1, wherein the pharmaceutically acceptable carrier is selected from the list consisting of a cream, an aerosol, an ointment, an oil, a liquid, a transdermal patch, a microneedle patch and an adhesive strip.

3. The composition of claim 1, wherein the heterologous nucleic acid encodes a heterologous gene product.

4. The composition of claim 1, wherein the heterologous nucleic acid encodes a bacterial toxin.

5. The composition of claim 1, wherein the heterologous nucleic acid encodes botulinum toxin.

6. The composition of claim 5, wherein the botulinum toxin is selected from the group consisting of botulinum toxin A, B, C₁, D, E, F and G.

7. The composition of claim 1, wherein the heterologous nucleic acid encodes an antisense nucleic acid sequence.

8. A method of delivering a heterologous nucleic acid to a mammalian target cell, comprising contacting the skin of the mammal with the composition of claim 1, under conditions whereby the baculovirus vector is introduced into the target cell.

9. A method of delivering a heterologous nucleic acid to a mammalian target cell through the epidermis of a mammal, comprising contacting the epidermis of the mammal with the composition of claim 1, under conditions whereby the baculovirus vector is transported through the epidermis and introduced into the target cell.

10. A method of delivering a heterologous nucleic acid to a mammalian target cell through the dermis of a mammal, comprising contacting the dermis of the mammal with the composition of claim 1, under conditions whereby the baculovirus vector is transported through the dermis and introduced into the target cell.

11. A method of delivering a heterologous nucleic acid to a mammalian target cell through the subcutaneous tissue of a mammal, comprising contacting the subcutaneous tissue of the mammal with the composition of claim 1, under conditions whereby the baculovirus vector is transported through the subcutaneous tissue and introduced into the target cell.

12. A method of expressing a heterologous nucleic acid in mammalian cell in a mammal to produce a cosmetic effect, comprising introducing into the mammalian cell a baculovirus vector, wherein said baculovirus vector comprises a promoter and a heterologous nucleic acid encoding a protein and/or antisense nucleic acid that produces a cosmetic effect.

13. The method according to claim 12, wherein the heterologous nucleic acid encodes a heterologous gene product.

14. The method according to claim 12, wherein the heterologous nucleic acid is an antisense nucleic acid.

15. The method according to claim 12, wherein the baculovirus is administered topically.

16. The method according to claim 12, wherein the baculovirus is administered subcutaneously.

17. A method of reducing skin wrinkles in a mammal, comprising contacting the composition of claim 5 with the skin of the mammal under conditions whereby the baculovirus vector is delivered to a target cell, heterologous nucleic acid is expressed to produce botulinum toxin and the botulinum toxin is delivered to a nerve cell associated with a muscle involved in a wrinkle, thereby reducing skin wrinkles in the mammal.

18. A method of tanning the skin of a mammal, comprising contacting the composition of claim 1 with the skin of the mammal, wherein the heterologous nucleic acid encodes a melanocyte modulatory protein and/or keratinocyte modulatory protein under conditions whereby the baculovirus vector is delivered to a target cell, the heterologous nucleic acid is expressed to produce the melanocyte modulatory protein and/or keratinocyte modulatory protein and the melanocyte modulatory protein and/or keratinocyte modulatory protein is delivered to melanocytes and/or keratinocytes, thereby tanning the skin of the mammal.

19. A method of reducing the incidence of acne in the skin of a mammal, comprising contacting the composition of claim 1 with the skin of the mammal, wherein the heterologous nucleic acid encodes androgen receptor and/or 5α reductase under conditions whereby the baculovirus vector is delivered to a target cell, the nucleic acid is expressed to produce the androgen receptor and/or 5α reductase and the androgen receptor and/or 5α reductase is delivered to sebaceous gland cells, thereby reducing the incidence of acne in the skin of a mammal.

20. A method of diminishing the appearance of a scar in the skin of a mammal, comprising contacting the composition of claim 1 with the skin of the mammal, wherein the heterologous nucleic acid encodes a [protein] under conditions whereby the baculovirus vector is delivered to a target cell, the heterologous nucleic acid is expressed to produce the protein and the protein is delivered to an effector cell, thereby diminishing the appearance of the scar.

21. A method of reducing a fat deposit in the skin of a mammal comprising contacting the composition of claim 1 with the skin of the mammal, wherein the heterologous nucleic acid encodes a protein selected from the group consisting of uncoupling protein-1, uncoupling protein-2, uncoupling protein-3, and β-andrenergic receptor 3, under conditions whereby the baculovirus vector is delivered to a target cell, the heterologous nucleic acid is expressed to produce the gene product of the heterologous nucleic acid and the gene product is delivered to an adipocyte, thereby reducing a fat deposit in the skin of the mammal.

22. A method of reducing perspiration in the skin of a mammal, comprising contacting the composition of claim 1 with the skin of the mammal, wherein the heterologous nucleic acid encodes sodium bicarbonate under conditions whereby the baculovirus vector is delivered to a target cell, the heterologous nucleic acid is expressed to produce sodium bicarbonate and sodium bicarbonate is delivered to neuronal cells, thereby reducing perspiration in the skin of a mammal.

23. A method of delivering vitamin E to a cell in the skin of a mammal comprising contacting the composition of claim 1 with the skin of the mammal, wherein the heterologous nucleic acid encodes vitamin E under conditions whereby the baculovirus vector is delivered to a target cell, the heterologous nucleic acid is expressed to produce vitamin E and vitamin E is delivered to an effector cell that metabolizes vitamin E, thereby delivering vitamin E to the skin of the mammal.

24. A method of reducing hair growth in the skin of a mammal comprising contacting the composition of claim 1 with the skin of the mammal, wherein the heterologous nucleic acid encodes a protein under conditions whereby the baculovirus vector is delivered to a target cell, the heterologous nucleic acid is expressed to produce the gene product of the heterologous nucleic acid and the gene product is delivered to an epithelial cell, thereby reducing the appearance of hair growth in the skin of the mammal.

25. A method of changing hair color in a mammal comprising contacting the composition of claim 1 with the skin of the mammal, wherein the heterologous nucleic acid encodes a protein under conditions whereby the baculovirus vector is delivered to a target cell, the heterologous nucleic acid is expressed to produce the gene product of the heterologous nucleic acid and the gene product is delivered to an epithelial cell, thereby increasing the hair color and/or growth of hair in the skin of the mammal.

26. A method of reducing signs of aging in the skin of a mammal comprising contacting the composition of claim 1 with the skin of the mammal, wherein the heterologous nucleic acid encodes a modulatory, protein under conditions whereby the baculovirus vector is delivered to a target cell, the heterologous nucleic acid is expressed to produce the gene product of the heterologous nucleic acid and the gene product is delivered to a keratinocyte, thereby reducing signs of aging in the skin of the mammal.

27. A method of inducing apoptosis in a cell in the skin of a mammal, comprising contacting the composition of claim 1 with the skin of the mammal, wherein the heterologous nucleic acid encodes a protein under conditions whereby the baculovirus vector is delivered to a target cell, the heterologous nucleic acid is expressed to produce the gene product of the heterologous nucleic acid and the gene product is delivered to an effector cell that cause apoptosis, thereby inducing apoptosis in a cell in the skin of a mammal.
